(19) United States
(12) Patent Application Publication
(10) Pub. No.: US 2025/0032133 A1
(43) Pub. Date: Jan. 30, 2025

(54) COMPUTER-ASSISTED NAVIGATION OF LOCK HOLE IN IMPLANT

(71) Applicant: ORTHOSOFT ULC, Montreal (CA)

(72) Inventor: Frederique DESBIENS BLAIS, Montreal (CA)

(21) Appl. No.: 18/770,738

(22) Filed: Jul. 12, 2024

Related U.S. Application Data
(60) Provisional application No. 63/515,262, filed on Jul. 24, 2023.

(51) Int. Cl. A61B 17/17; A61B 34/10; A61B 90/00

(52) U.S. Cl. CPC A61B 17/1717; A61B 17/1703; A61B 17/1728; A61B 34/10; A61B 90/37; A61B 2034/104; A61B 2034/105; A61B 2090/365; A61B 2090/373

(57) ABSTRACT

A system for tracking a surgical implant relative to a bone in computer-assisted surgery, may have a processing unit; and a non-transitory computer-readable memory communicatively coupled to the processing unit and comprising computer-readable program instructions executable by the processing unit for: obtaining a virtual model of the surgical implant, the virtual model including at least one landmark of the surgical implant, the at least one landmark being configured to be inside the bone; tracking the surgical implant, via an optical non-radiographic tracking device, as the surgical implant is inserted into the bone; calculating a location of the at least one landmark relative to the bone using the virtual model of the surgical implant and tracking data from the tracking of the surgical implant; and outputting the location of the at least one landmark relative to the bone.

### CROSS-REFERENCE TO RELATED APPLICATION

The present application claims the priority of U.S. Patent Application No. 63/515,262, filed on Jul. 24, 2023, and incorporated herein by reference in its entirety.

### TECHNICAL FIELD

The present application relates to implant tracking in computer-assisted orthopedic surgery and/or in robotized computer-assisted surgery.

### BACKGROUND OF THE ART

The navigation of surgical instruments or tools is an integral part of computer-assisted surgery (hereinafter “CAS”). The tools are navigated, i.e., tracked for position and/or orientation, in such a way that relative information pertaining to bodily parts is obtained. The information may be used in various interventions (e.g., orthopedic surgery, trauma surgery, neurological surgery) with respect to the body, such as bone alterations, implant positioning, incisions and the like during surgery.

In some circumstances, some implants must penetrate bones, such that the tracking of the implant becomes more difficult in the absence of visibility. For example, an intramedullary nail, also known as an intramedullary rod, is a metallic implant that is forced into the intramedullary canal (a.k.a., medullary canal or cavity) of the bone, in the case of a long bone fracture (e.g., tibia fracture, femur fracture). The implant contributes to the load bearing of the bone, by defining a structure holding the bone together. However, one challenge with the installation of such implants is its lack of visibility once inserted into the intramedullary canal.

Likewise, in similar circumstances, for minimally invasive surgeries, it is desirable to limit incision size and position implants such as plates along the bones, where the plates are inserted via a small incision and slid along the bone. Thus, plates may not be visible because they are concealed under the skin. Then, there remains a challenge in finding bolts holes on the plate.

### SUMMARY

In accordance with a first aspect of the present disclosure, there is provided a system for tracking a surgical implant relative to a bone in computer-assisted surgery, comprising: a processing unit; and a non-transitory computer-readable memory communicatively coupled to the processing unit and comprising computer-readable program instructions executable by the processing unit for: obtaining a virtual model of the surgical implant, the virtual model including at least one landmark of the surgical implant, the at least one landmark being configured to be inside the bone; tracking the surgical implant, via an optical non-radiographic tracking device, as the surgical implant is inserted into the bone; calculating a location of the at least one landmark relative to the bone using the virtual model of the surgical implant and tracking data from the tracking of the surgical implant; and outputting the location of the at least one landmark relative to the bone.

### DESCRIPTION OF THE DRAWINGS

FIG. 1 is a block diagram of a computer-assisted surgery (CAS) system with optional head-mounted navigation in accordance a variant with the present disclosure;

FIG. 2 is a perspective view and block diagram of a head-mounted device in accordance with another variant of the present disclosure;

FIG. 3A is a view of an exemplary femoral intramedullary nail navigated with the CAS system of FIG. 1;

FIG. 3B is a view of an exemplary tibial intramedullary nail navigated with the CAS system of FIG. 1;

FIG. 4 is a perspective view of an exemplary plate navigated with the CAS system of FIG. 1;

FIG. 5 is a perspective view of a pair of plates navigated with the CAS system of FIG. 1;

FIG. 6 is a perspective view of the CAS system of FIG. 1 during a resection of the tibia; and

FIG. 7 is a perspective view of the CAS system of FIG. 1 during a resection of the tibia, with assistance from a robot arm.

### DETAILED DESCRIPTION

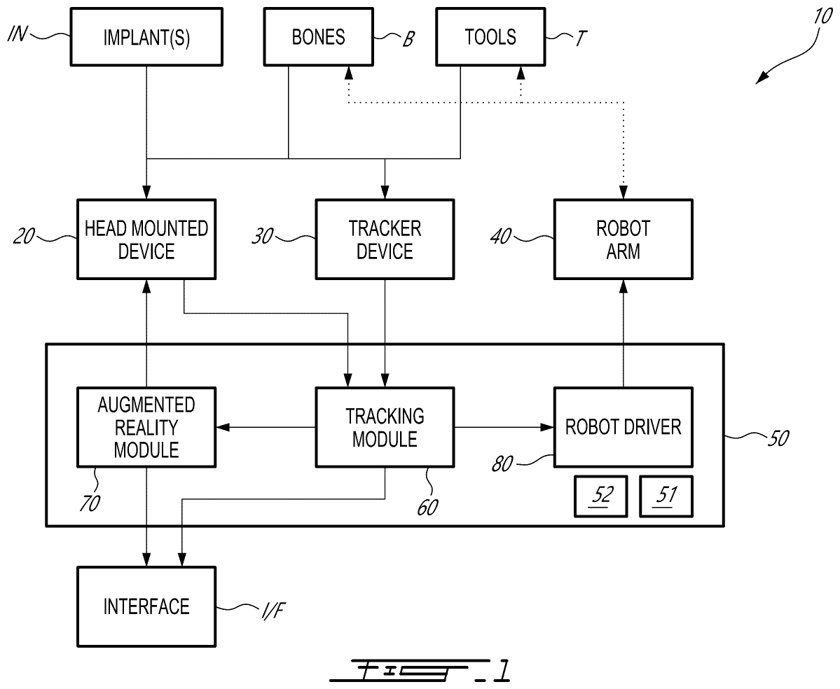

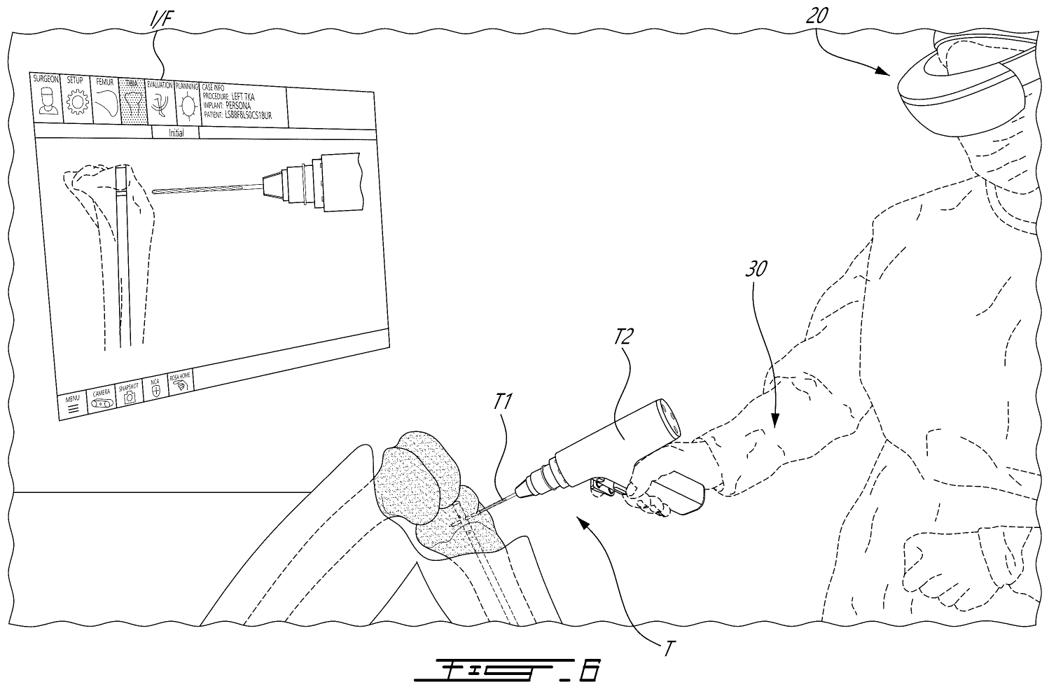

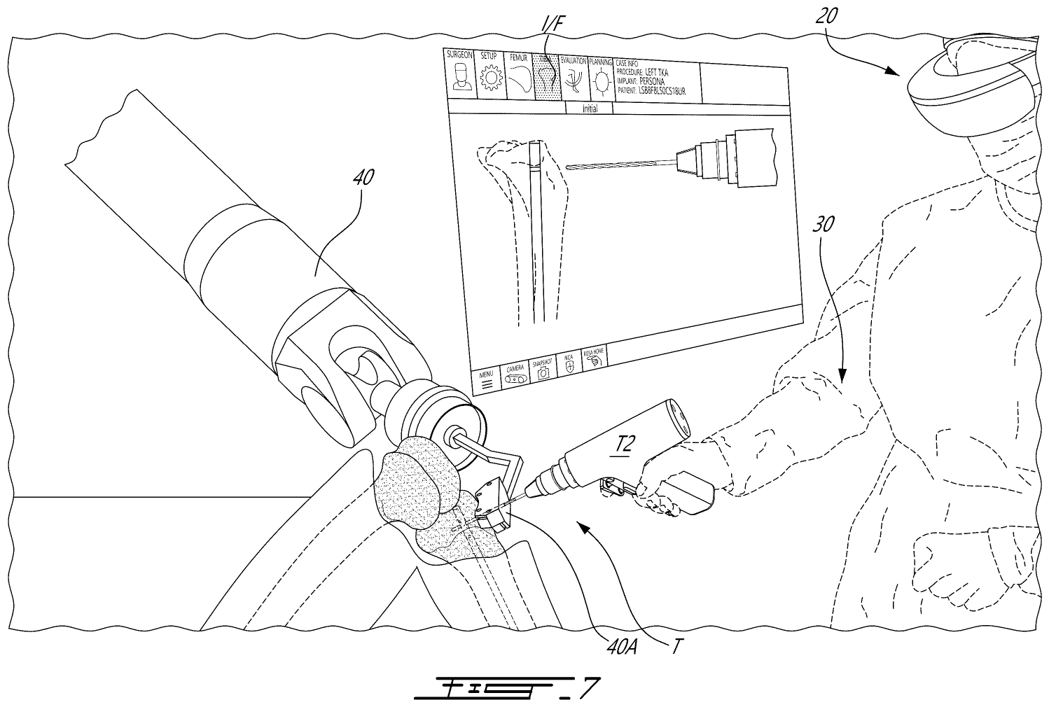

Referring to the drawings and more particularly to FIG. 1, computer-assisted surgery (CAS) system with head-mounted navigation with optional combined tracking is generally shown at 10, and is used to provide surgery assistance to an operator in screw hole localization, when such screw holes may not be visible as concealed by bone and/or soft tissue. For example, the CAS system 10 could be used to assist in implanting an intramedullary nail in an intramedullary canal of a bone, such as a long bone (e.g., femur, tibia, humerus). The expression “intramedullary nail” is used herein, but other expressions therefor include intramedullary rod, intramedullary implant. Moreover, the expression intramedullary may also be referred to as medullary. The CAS system 10 could also be used to track a position and orientation of one or more plates, known as orthopedic plates, internal fixation plates, bone plates, trauma plates, etc. The CAS system 10 may be used to assist an operator wearing a head-mounted device with display in performing the afore-mentioned maneuvers and/or other surgical maneuvers on a patient, such as surgical maneuvers associated with orthopedic surgery, including pre-operative analysis of range of motion, and implant assessment planning, as described hereinafter. In FIGS. 6 and 7, the system 10 is shown relative to a patient's knee joint in supine decubitus, but only as an example. The system 10 could be used for other body parts, including non-exhaustively hip joint, spine, and shoulder bones, or in other types of surgery.

The CAS system 10 may be robotized in a variant, and has, may have or may be used with a head-mounted device or tracking device 20, a tracking device 30, a robot arm 40, a CAS controller 50, a tracking module 60, an augmented reality module 70, and a robot driver 80, or any combination thereof:
The head-mounted device 20 is worn by an operator, such as by the surgeon performing surgery, and may be referred to as head-mounted tracking device 20 as it has the capacity to capture images, such as in video format. The head-mounted device 20 may have a display screen to provide data to the wearer, though this may be optional in an embodiment. For simplicity, the expressions head-mounted tracking device 20 and head-mounted device 20 are used interchangeably in the present disclosure and figures. The head-mounted tracking device 20 may be used to provide a display in augmented/mixed and/or virtual reality to a user. The head-mounted tracking device 20 may also be tasked with taking images of the surgery, with the images being used for the tracking of patient tissue (such as bones) and tools, for instance as a video feed. The head-mounted tracking device 20 may also be used as an interface by which an operator may communicate commands to the CAS system 10.
As an alternative to the head-mounted device 20, the tracking device 30 may optionally be used to track the patient tissue, instruments, and the robot arm 40. For example, the tracking device 30 may complement the tracking performed with the imaging done with the head-mounted tracking device 20, and may hence be referred to as a secondary tracking device. The tracking device 30 may employ camera technology similar to that of the head-mounted tracking device 20, such as depth cameras, with optional pattern projector, as described below, or may be a different imaging technology, to provide its video feed. The tracking device 30 may be said to be stationary. The tracking device 30 may be the primary tracking device 30, if the head-mounted tracking device 20 is absent or temporarily out of the line of the surgical scene, or with the head-mounted tracking device 20 providing complementary tracking capability. The tracking device 30 may use ultrasounds as well. The tracking device 30 may operate with optical trackers on the objects, such as those used in a Navitrack® system, but other types of trackers could be used, including QR codes, AprilTags, 3D trackers having a wider range of visibility (cube or other prism), etc.
The robot arm 40 may optionally be present as the working end of the system 10—the CAS system 10 could also be non robotic-, and may be used to guide or to perform bone alterations as planned by an operator and/or the CAS controller 50 and as controlled by the CAS controller 50, or may be used to support a tool T that is operated by a surgeon. The robot arm 40 may also be configured for collaborative/cooperative mode in which the operator may manipulate the robot arm 40. For example, the tooling end, also known as end effector, and/or tool T at the tooling end may be manipulated by the operator while supported by the robot arm 40;
The CAS controller 50 includes the processor(s) and appropriate hardware and software to run a computer-assisted surgery procedure in accordance with one or more workflows. The CAS controller 50 may include or operate the tracking module 60, the augmented reality module 70, and/or the robot driver 80 if present. Moreover, as described hereinafter, the CAS controller 50 may also drive the robot arm 40 through a planned surgical procedure, if the robot arm 40 is present;
The tracking module 60 is tasked with determining the position and/or orientation of the various relevant objects during the surgery procedure, such as the bone(s) and tool(s), using data acquired by the head-mounted tracking device 20 (e.g., video feed) if present, the tracking device 30 if present. The position and/or orientation may be used by the CAS controller 50 to control the robot arm 40;
The augmented reality module 70 (a.k.a., mixed reality module) is provided to produce an augmented reality output to the operator, for instance for display in the head-mounted tracking device 20. The augmented reality module 70 may also produce other types of outputs, including a virtual reality output. The augmented reality module 70 may provide its output to displays other than head-mounted or wearable displays. For example, the augmented reality module 70 may produce an output for display on monitors of the CAS system 10, shown in FIG. 1 as interface I/F;
The robot driver 80 is tasked with powering or controlling the various joints of the robot arm 40, if present, based on operator demands or on surgery planning.

Other components, devices, systems, may be present, such as surgical instruments and tools T, the interfaces I/F such as displays, screens, computer station, servers, and the like etc.

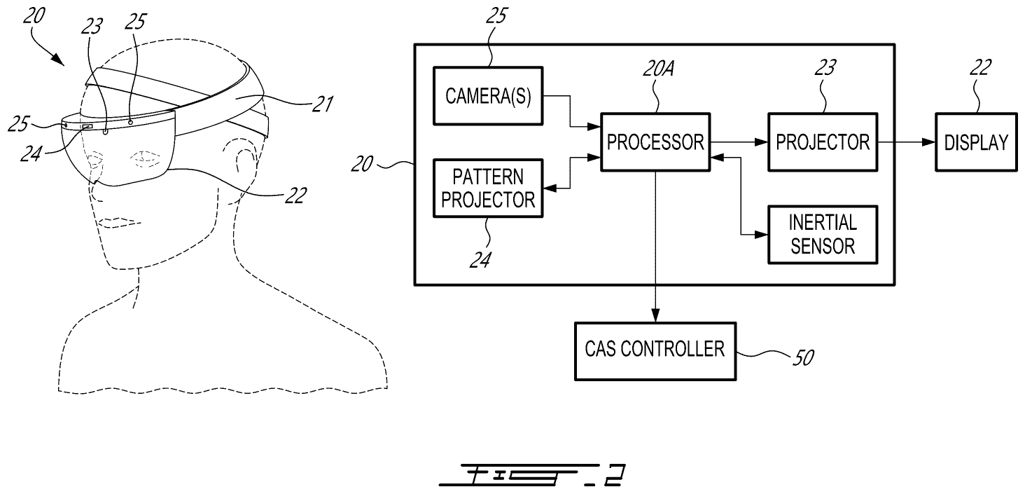

Referring to FIG. 2, a schematic example of the head-mounted tracking device 20 is provided. The head-mounted tracking device 20 may be as described in U.S. Pat. No. 10,687,568, the contents of which are incorporated herein by reference, or may have other configurations. The head-mounted tracking device 20 described as a surgical helmet assembly in U.S. Pat. No. 10,687,568 is well suited to be used in an augmented reality setting by its configuration. The head-mounted tracking device 20 may have a head enclosure 21 shaped to encircle a head of an operator. The head enclosure 21 may be straps, a rim, a helmet, etc. A face shield 22 may be mounted to a forehead or brow region of the head enclosure 21. The face shield 22 may be transparent to allow see-through vision by a user, but with the option of serving as a screen for augmented reality. Other components of the head-mounted tracking device 20 may include stabilizers, head band, a ventilation system with fan and vents, a light source, a rechargeable power source (e.g., a battery) etc.

The head-mounted tracking device 20 may consequently include a processor 20A and components to produce a mixed reality session. For instance, the head-mounted tracking device 20 may have an integrated projector 23 that may project data on the face shield 22, in a manner described below. Alternatively, the face shield 22 may be a screen having the ability to display images. As an example, the head-mounted tracking device 20 may be a Hololens®. In an embodiment, the face shield 22 is a display-like unit of the type that may be used in virtual reality, with camera(s) therein to create a mixed reality output using camera footage, such as an Oculus Rift®, smartphone with head support, etc, hologram projection in augmented reality. The head-mounted tracking device 20 may include one or more orientation sensors, such as inertial sensor unit(s) (e.g., shown as 30), for an orientation of the head-mounted tracking device 20 to be known and tracked.

According to an embodiment, the head-mounted tracking device 20 is equipped to perform optical tracking of an implant IN, such as the intramedullary implant or orthopedic plate, patient tissue B, instruments T and/or robot arm 40, from a point of view (POV) of the operator. The head-mounted tracking device 20 may therefore have one or more imaging devices or apparatuses, to capture video images of a scene, i.e., moving visual images, a sequence of images over time. In a variant, the video images are light backscatter (a.k.a. backscattered radiation) used to track objects. In the present disclosure, the head-mounted tracking device 20 may be used to track tools and bones so as to provide navigation data in mixed reality to guide an operator based on surgery planning. Backscattered radiation can also be used for acquisition of 3D surface geometries of bones and tools.

The head-mounted tracking device 20 may produce structured light illumination for tracking objects with structured light 3D imaging. In structured light illumination, a portion of the objects is illuminated with one or multiple patterns from a pattern projector 24 or like light source. The pattern projector 24 includes infrared light projection. Structured light 3D imaging is based on the fact that a projection of a line of light from the pattern projector 24 onto a 3D shaped surface produces a line of illumination that appears distorted as viewed from perspectives other than that of the pattern projector 24. Accordingly, imaging such a distorted line of illumination allows a geometric reconstruction of the 3D shaped surface. Imaging of the distorted line of illumination is generally performed using one or more cameras 25 (including appropriate components such as e.g., lens(es), aperture, image sensor such as CCD, image processor) which are spaced apart from the pattern projector 24 so as to provide such different perspectives, e.g., triangulation perspective. In some embodiments, the pattern projector 24 is configured to project a structured light grid pattern including many lines at once as this allows the simultaneous acquisition of a multitude of samples on an increased area. In these embodiments, it may be convenient to use a pattern of parallel lines. However, other variants of structured light projection can be used in some other embodiments.

The structured light grid pattern can be projected onto the surface(s) to track using the pattern projector 24. In some embodiments, the structured light grid pattern can be produced by incoherent light projection, e.g., using a digital video projector, wherein the patterns are typically generated by propagating light through a digital light modulator. Examples of digital light projection technologies include transmissive liquid crystal, reflective liquid crystal on silicon (LCOS) and digital light processing (DLP) modulators. In these embodiments, the resolution of the structured light grid pattern can be limited by the size of the emitting pixels of the digital projector. Moreover, patterns generated by such digital display projectors may have small discontinuities due to the pixel boundaries in the projector. However, these discontinuities are generally sufficiently small that they are insignificant in the presence of a slight defocus. In some other embodiments, the structured light grid pattern can be produced by laser interference. For instance, in such embodiments, two or more laser beams can be interfered with one another to produce the structured light grid pattern wherein different pattern sizes can be obtained by changing the relative angle between the laser beams.

The pattern projector 24 may emit light that is inside or outside the visible region of the electromagnetic spectrum. For instance, in some embodiments, the emitted light can be in the ultraviolet region and/or the infrared region of the electromagnetic spectrum such as to be imperceptible to the eyes of the medical personnel. In these embodiments, however, the medical personnel may be required to wear protective glasses to protect their eyes from such invisible radiations, and the face shield 22 may have protective capacity as well. As alternatives to structured light, the head-mounted tracking device 20 may also operate with laser rangefinder technology or triangulation, as a few examples among others.

The head-mounted tracking device 20 may consequently include the cameras 25 to acquire backscatter images of the illuminated portion of objects. Hence, the cameras 25 capture the pattern projected onto the portions of the object. The cameras 25 are adapted to detect radiations in a region of the electromagnetic spectrum that corresponds to that of the patterns generated by the light projector 24. As described hereinafter, the known light pattern characteristics and known orientation of the pattern projector 24 relative to the cameras 25, are used by the tracking module 60 to generate a 3D geometry of the illuminated portions, using the backscatter images captured by the camera(s) 25. Although a single camera spaced form the pattern projector 24 can be used, using more than one camera 25 may increase the field of view and increase surface coverage, or precision via triangulation. The head-mounted tracking device 20 is shown as having a pair of cameras 25 is used.

The head-mounted tracking device 20 may also have one or more filters integrated into either or both of the cameras 25 to filter out predetermined regions or spectral bands of the electromagnetic spectrum. The filter can be removably or fixedly mounted in front of any given camera 25. For example, the filter can be slidably movable into and out of the optical path of the cameras 25, manually or in an automated fashion. In some other embodiments, multiple filters may be periodically positioned in front of a given camera in order to acquire spectrally resolved images with different spectral ranges at different moments in time, thereby providing time dependent spectral multiplexing. Such an embodiment may be achieved, for example, by positioning the multiple filters in a filter wheel that is controllably rotated to bring each filter in the filter wheel into the optical path of the given one of the camera 25 in a sequential manner.

In some embodiments, the filter can allow transmittance of only some predetermined spectral features of objects within the field of view, captured either simultaneously by the head-mounted tracking device 20 or separately by the secondary tracking device 90, so as to serve as additional features that can be extracted to improve accuracy and speed of registration.

More specifically, the filter can be used to provide a maximum contrast between different materials which can improve the imaging process and more specifically the soft tissue identification process. For example, in some embodiments, the filter can be used to filter out bands that are common to backscattered radiation from typical soft tissue items, the surgical structure of interest, and the surgical tool(s) such that backscattered radiation of high contrast between soft tissue items, surgical structure and surgical tools can be acquired. Additionally, or alternatively, where white light illumination is used, the filter can include band pass filters configured to let pass only some spectral bands of interest. For instance, the filter can be configured to let pass spectral bands associated with backscattering or reflection caused by the bones, the soft tissue while filtering out spectral bands associated with specifically colored items such as tools, gloves and the like within the surgical field of view. Other methods for achieving spectrally selective detection, including employing spectrally narrow emitters, spectrally filtering a broadband emitter, and/or spectrally filtering a broadband imaging detector (e.g., the camera 25), can also be used. Another light source may also be provided on the head-mounted tracking device 20, for a secondary tracking option, as detailed below. It is considered to apply distinctive coatings on the parts to be tracked, such as the bone and the tool, to increase their contrast relative to the surrounding soft tissue.

In accordance with another embodiment, the head-mounted tracking device 20 may include a 3D camera(s), also shown as 25, to perform range imaging, and hence determine position data from the captured images during tracking—FIG. 2 showing two of such cameras 25 to enhance a depth perception. The expression 3D camera is used to describe the camera's capability of providing range data for the objects in the image or like footage it captures, but the 3D camera may or may not produce 3D renderings of the objects it captures. In contrast to structured light 3D imaging, range tracking does not seek specific illumination patterns in distance calculations, but relies instead on the images themselves and the 3D camera's capacity to determine the distance of points of objects in the images. Stated differently, the 3D camera for ranging performs non-structured light ranging, and the expression “ranging” is used herein to designate such non-structured light ranging. Such range tracking requires that the 3D camera be calibrated to achieve suitable precision and accuracy of tracking. In order to be calibrated, the head-mounted tracking device 20 may use a known visual pattern in a calibration performed in situ, at the start of the tracking, and optionally updated punctually or continuously throughout the tracking. The calibration is necessary to update the camera acquisition parameters due to possible lens distortion (e.g., radial, rotational distortion), and hence to rectify image distortion to ensure the range accuracy. Moreover, as described herein, tracking tokens with recognizable patterns (e.g., QR codes) may be used, with the patterns being used to determine a point of view (POV) of the cameras 25 of the head-mounted tracking device 20, via perspective deformation.

In a variant, the head-mounted tracking device 20 only has imaging capacity, for instance through cameras 25 (of any type described above), optionally pattern projector 24, without other components, such as face shield 22, etc.

Referring to FIGS. 1 and 7, if present, the robot arm 40 may stand from a base, for instance in a fixed relation relative to the operating-room (OR) table supporting the patient, whether it is attached or detached from the table. The robot arm 40 has a plurality of joints and links, of any appropriate form, to support a tool T that interfaces with the patient. The end effector or tool head may indeed optionally incorporate a force/torque sensor for collaborative/cooperative control mode, in which an operator manipulates the tool T at the end of the robot arm 40. The robot arm 40 is shown being a serial mechanism, arranged for the tool head to be displaceable in a desired number of degrees of freedom (DOF). For example, the robot arm 40 controls 6-DOF movements of the tool head, i.e., X, Y, Z in the coordinate system, and pitch, roll and yaw. Fewer or additional DOFs may be present. For simplicity, only a generic illustration of the joints and links is provided, but more joints of different types may be present to move the tool head in the manner described above. The joints are powered for the robot arm 40 to move as controlled by the CAS controller 50 in the six DOFs, and in such a way that the position and orientation of the tool head in the coordinate system may be known, for instance by readings from encoders on the various joints. Therefore, the powering of the joints is such that the tool head of the robot arm 40 may execute precise movements, such as moving along a single direction in one translation DOF, or being restricted to moving along a plane, among possibilities. Such robot arms 40 are known, for instance as described in U.S. patent application Ser. No. 11/610,728, and incorporated herein by reference. The head-mounted tracking device 20 and/or tracking device 30 may be used for the tracking of the end effector of the robot arm 10, or other systems such as inertial sensor systems, e.g., an inertial sensor unit may be on the robot arm 40.

Still referring to FIG. 1, the CAS controller 50 is shown in greater detail relative to the other components of the robotized CAS system 10. The CAS controller 50 has a processor unit 51 (one or more processors) and a non-transitory computer-readable memory 52 communicatively coupled to the processing unit 51 and configured for executing computer-readable program instructions executable by the processing unit 51 to perform some functions, such as tracking the patient tissue and tools, using the camera feed from the head-mounted tracking device 20 and/or from the tracking device 90, and the readings from the inertial sensor unit(s) 30. The CAS controller 50 may also control the movement of the robot arm 40. The CAS system 10 may comprise various types of interfaces I/F, for the information to be provided to the operator. In addition to the head-mounted tracking device 20, the interfaces I/F may include a monitor and/or screens including wireless portable devices (e.g., phones, tablets), audio guidance, LED displays, among many other possibilities. For example, the interface D includes a graphic-user interface (GUI) operated by the system 10. The CAS controller 50 may also display images captured by the cameras 25 of the head-mounted tracking device 20 and/or tracking device 90, for instance to be used in the collaborative/cooperative control mode of the system 10, or for visual supervision by the operator of the system 10, with augmented reality for example. The CAS controller 50 may drive the robot arm 40, if present, in performing the surgical procedure based on the surgery planning achieved pre-operatively. The CAS controller 50 may run various modules, in the form of algorithms, code, non-transient executable instructions, etc, in order to operate the CAS system 10 in the manner described herein. The CAS controller 50 may be part of any suitable processor unit(s), such as a personal computer or computers including laptops and desktops, tablets, server, cloud, etc.

The tracking module 60 may be a subpart of the CAS controller 50, or an independent module or system. The tracking module 60 receives from the head-mounted tracking device 20 and the tracking device 90 (if present) the video feed of the surgical scene, e.g., as backscatter images of the objects. The tracking module 60 may also concurrently receive tracking data (e.g., orientation data) from the inertial sensor unit(s) 30. In an embodiment, as the system 10 performs real-time tracking, the video images and the orientation data are synchronized, as they are obtained and processed simultaneously. Other processing may be performed to ensure that the video footage and the orientation data are synchronized.

The tracking module 60 processes the video images to track one or more objects, such as a bone, an instrument, etc. The tracking module 60 may determine the relative position of the objects, and segment the objects within the video images. In a variant, the tracking module 60 may process the video images to track a given portion of an object, that may be referred to as a landmark. The landmark may be different parts of the objects, objects on the objects, such as tracking tokens with recognizable patterns, etc.

The tracking module 60 may also be provided with models of the objects to be tracked. For example, the tracking module 60 may track bones, implants IN and tools, and hence uses virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual implant models and/or tool models may be provided by the implant/tool manufacturer, or may also be generated in any appropriate way so as to be a virtual 3D representation of the tool(s).

In a variant, the tracking module 60 may generate 3D models using the video images. For example, if the tracking module 60 can have video images of a tool, from 360 degrees, it may generate a 3D model that can be used for subsequent tracking. This intraoperative model may or may not be matched with pre-existing or pre-operative model of the tool and/or of the implant.

Additional data may also be available, such as tool orientation (e.g., axis data and geometry). By having access to bone, implant and tool models, the tracking module 60 may recognize an object in the image processing and/or may obtain additional information, such as the axes related to bones or tools. The image processing by the tracking module 60 may be assisted by the presence of the models, as the tracking module 60 may match objects from the video images with the virtual models.

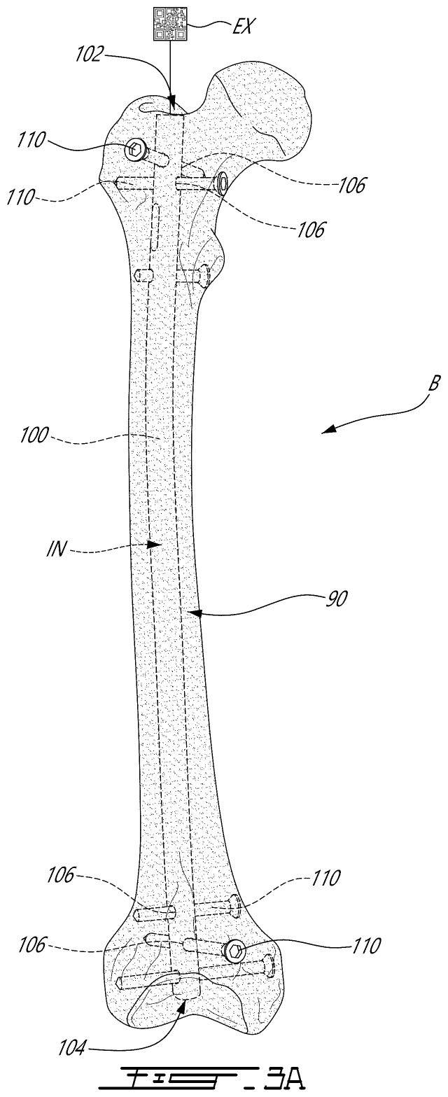

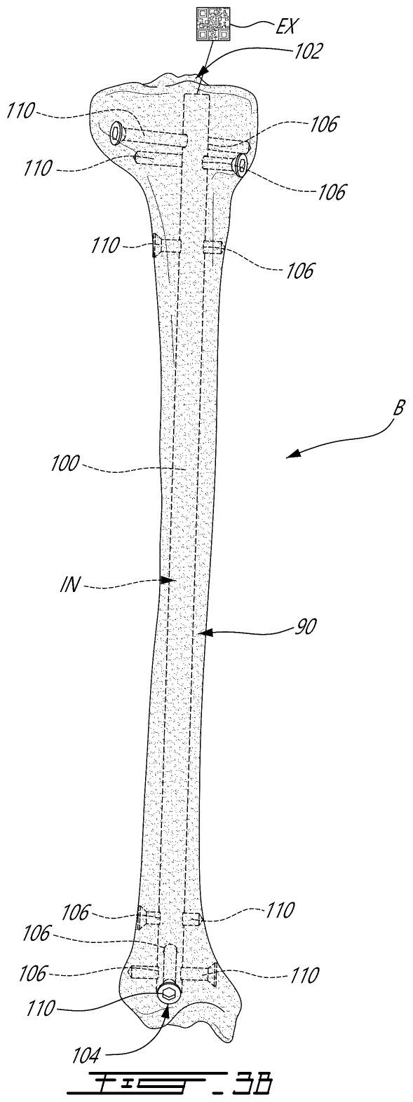

For example, two distinct implants IN inside bones N are shown in FIGS. 3A and 3B. FIG. 3A shows implant IN inside a femur bone B, while FIG. 3B shows the implant IN inside a tibia bone B. Each bone has an intramedullary canal 90 through which an implant IN (illustratively an intramedullary nail) is inserted. The intramedullary nail IN may be defined as having an elongated rod or shaft 100 spanning across fractures, with locking and/or stabilizing screws or like fasteners at the opposed ends of the shaft 100. The implant IN is thus defined by the shaft 100 extending between a proximal end 102 and a distal end 104. One or more landmark locations are found at the proximal end 102 and the distal end 104 of the implant IN. In the shown embodiment, the landmark locations are through holes, such as threaded screw holes 106, also referred to as locking holes 106 (also known as screw holes), extending through the shaft 100 at the proximal end 102 and the distal end 104 of the implant IN, in an orientation that may be generally transverse to the longitudinal axis of the implant IN. The number of locking holes 106, as well as their position and orientation (collectively referred to as their location) may vary. Fasteners 110 such as lag screws, stabilizing screws and/or locking screws are insertable through the locking holes 106 to secure the implant IN to the bone B.

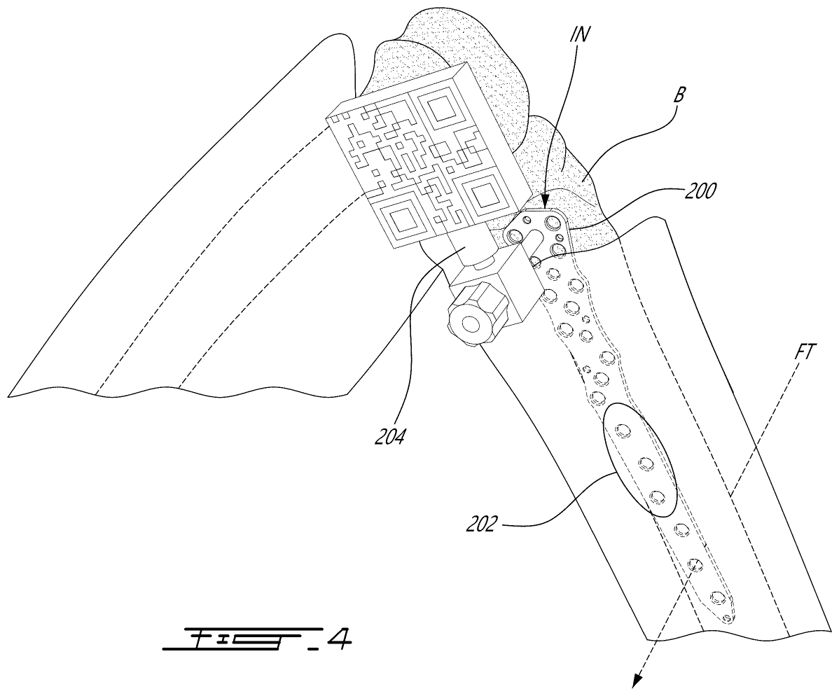

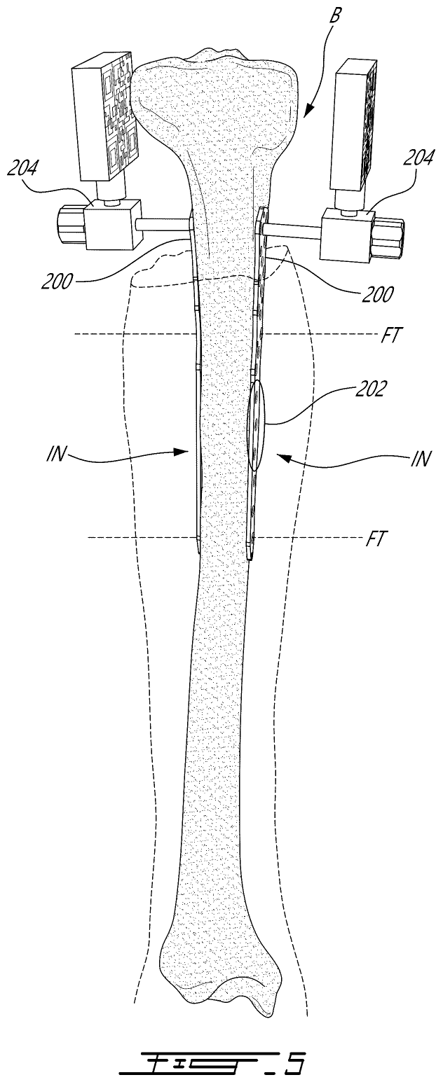

As another example, referring to FIGS. 4 and 5, the implants IN are orthopedic plates, positioned on the surface of the bone and having a substantial portion under soft tissue, as the orthopedic plates IN may be slid into location. In both FIGS. 4 and 5, the implants IN are on a surface of a tibia bone B, but plates IN could be used with other bones. In FIG. 4, a single plate IN is shown, while in FIG. 5, a pair of plates IN are shown, with the plates IN being interlocked during the surgical procedure. The plate IN may be defined as having a plate body 200 having a plurality of holes 202. A bracket 204 may optionally be present to support a trackable marker, such as a QR code as an example. The bracket 204 may be present during implanting, and removed thereafter. The plate body 200 may be elongated and slender, to be used with a long bone such as the tibia. In a variant, the plate body 200 may have a known 3D geometry. In another variant, the plate body 200 may be deformed before insertion, for the plate body 200 to be given a shape emulating curvatures in the bone. The deforming may be done intraoperatively, for instance by manually applying forces. In yet another embodiment, the plate body 200 is a patient-specific implant, manufactured based on patient imaging. The plate body 200 spans across fractures, with locking and/or stabilizing screws or like fasteners penetrating the bone from the opposite side, to pass through the bone and to reach a screw hole 202. There may therefore be more than one such locking screw, as the operator may be given a choice. The screw holes 202 may be known as the landmark locations, and may be referred to as threaded screw holes, locking holes, etc. The number of locking holes 202, as well as their position and orientation (collectively referred to as their location) may vary. Fasteners such as lag screws, stabilizing screws and/or locking screws are insertable through the bones to reach the holes 202 by following a trajectory FT and hence may contribute in securing the implant IN to the bone B. In the case of FIG. 5, the plates 200 on opposite sides of the bone B must be aligned with one another, for screws to have their opposed ends operatively engaged to respective holes 202 in plate 200, with a more central portion of the screws passing through the bone B. As described below, the CAS system 10 provides such guidance, by providing the trajectory FT to the operator.

In a variant, the objects used as landmarks are parts of the bone, implant and of the tool that are visible from the head-mounted tracking device 20 and/or from the tracking device 30. Stated differently, as the operator has a direct and proximal view of the surgical site, e.g., the bone being drilled, the implant being inserted in the intramedullary canal or the plates being positioned along the bone, and the tool performing a drilling action, the footage from the POV of the head-mounted tracking device 20 is used by the tracking module 60 to navigate the tool T and implant IN relative to the bone B. Despite the variation in POV of the camera(s) 25, the tracking module 60 using the known dimensions of a landmark to track the objects in a referential system. The landmark may be an extension EX (FIGS. 3A and 3B), may be a bracket 204 (FIGS. 4 and 5), or may be a visible end of the implant IN or tool T, if a sufficient large and distinct part is visible. The body of the implants IN and/or tools T may for example be used as a basis for the tracking, as explained above, by way of the model of the implant IN (e.g., IN in FIGS. 3A and 3B), which model can be a three-dimensional model, that may result from scanning and/or from obtaining a manufacturer's file. If an implant is deformed intraoperatively, such as is the case with the plates bodies 200 of FIGS. 4 and 5 in some circumstances, it may be necessary to calibrate the altered geometry before insertion. For example, the calibration may be done using the bracket 204, and a tool inserted in one or more of the holes 202, for the position and orientation of the holes 202 to be known relative to the bracket 204 (or other tracking feature). Even when parts of the implant IN or bone B are concealed, or not in the line of sight, the use of the models of the implant IN and bone B may enable to provide images of hidden parts of the implant IN and bone B, such as on the GUls of FIGS. 6 and 7. More particularly, there may be enough of the surface of the implant IN visible to map the visible surface with the model of the implant IN. It is also considered to connect a tracker at the end of the implant IN, which tracker device is visible by the tracking system or camera. For example, the tracker is a removable extension EX (e.g., FIG. 3A, but applicable to all figures) to the implant IN and has a known geometry and a known geometrical relation relative to the implant IN, and the bracket 204 in FIGS. 4 and 5. Thus, even if the implant IN is fully inserted in the bone B, the tracking module 60 can know the precise spatial location of the implant IN by optically (and non-radiographically) seeing the removable extension EX, or the drilling trajectory FT for subsequent insertion of fasteners that will screwingly engaged with a fastener hole(s) 202.

Optionally, it is considered to provide specific detectable landmarks on the implant IN, tool(s) or bones to ensure the detectable landmarks will be properly imaged and detected by the tracking module 60. For example, tokens with given patterns, such as QR-code like labels, AprilTags, etc, may be provided on the objects, such as on the bones or on the tools, such as on the brackets 204 or extensions EX described above. In an embodiment, the tokens have an image that may be preprogrammed, or whose dimensions, are known or accessible by the tracking module 60. Accordingly, by seeing such tokens in the video images, the tracking module 60 may locate the objects in a spatial coordinate system. Again, the POV for the video images may move, with the objects serving as a referential for the tracking. The referential system may be that of the robot arm 20 (if present), with the camera tracking providing positional information for the referential system.

In matching the recognizable pattern and/or the 3D geometry to the implant models, bone models and tool models with the video images, the tracking module 60 may reduce its computation using different strategies. The bone model(s) B may have higher resolution for the parts of the bone that will be altered during surgery. The remainder of the bone may be limited to information on landmarks, such as axis orientation, center of rotation, midpoints, etc. A similar approach may be taken for the tool models C, with the focus and higher detail resolution being on parts of the tools that come into contact with the bone. The bone model(s) B may also include implants that are already in the bone, for example from a past procedure. The CAS system 10 may thus have the capacity to guide the procedure in determining any potential interference between implant IN and existing implant, to avoid damaging existing implants. For example, in the case of the plates IN of FIGS. 4 and 5, this may mean suggesting the use of some of the holes 202 and blocking the use of other holes 202, based on interference with an implant.

Moreover, considering that the camera(s) 25 may interrupt its line of sight with the object, the video feed or like tracking from the tracker device 30 may complement that from the camera(s) 25 and/or supersede the video feed from the camera(s) 25. In another embodiment, the tracker device 30 is the primary tracking camera using any of the technologies described above for the head-mounted tracking device 20. Therefore, the tracking module 60 continuously updates the position and/or orientation of the patient bones and tools in the coordinate system using the video feed from the camera(s) 25 and/or tracking device 30 to track objects in position and orientation.

In an embodiment with structured light projection, the tracking module 60 receives the backscatter images from the camera(s) 25 or from the tracking device 90, as a result of the structured light projection from the projector 24. In another embodiment, the tracking module 60 receives the video images from the camera 25 in a depth camera configuration, and may take steps to calibrate the camera(s) 25 or tracking device 90 for ranging to be done from the acquired images. An initial calibration may be done using a calibration pattern, such as that in tokens. The calibration pattern is placed in the light of sight of the camera(s) 25 or tracking device 90 such that it is imaged. The calibration pattern is any appropriate shape and configuration, but may be a planar recognizable pattern with high contrast, or given landmarks of a bone, or geometry of tool. Other items can be used for the calibration, including the body of a tool T, whose geometry may be programmed into or may be accessed by the tracking module 60. The tracking module 60 stores a virtual version of the calibration pattern, including precise geometrical data of the calibration pattern. The tracking module 60 therefore performs a correspondence between imaged and virtual calibration patterns. The correspondence may entail calculating the mapping function between landmarks on the planar imaged calibration pattern and the virtual calibration pattern. This may include a projection of the calibration patterns on one another to determine the distortion characteristics of the images of the camera(s) 25 or tracking device 90, until the rectification values are determined by the tracking module 60 to correct the images of camera. This calibration may be repeated punctually through the procedure, for instance based on the camera updating requirements. It may require that the camera be used in conjunction with a calibration reflective surface whose position and orientation relative to the camera is known. The calibration may be automatically performed by the CAS system 10.

The tracking module 60 may therefore perform a 3D geometry image processing, using the known patterns of structured light, or calibrated camera images, video feed, etc, along with the known shape of the virtual bone model(s) and/or tool model(s), optionally with QR tokens, and generate 3D images from the tracking, using for examples the pre-operative models. Moreover, a generated 3D geometry may be located in the X, Y, Z, coordinate system using the tracking of landmarks on the bones or tools to set the coordinate system on the bones. Therefore, the tracking module 60 may generates an image or 3D geometry of the landmarks on the object(s) being illuminated. Then, using the virtual models and/or of the bone(s) and tool(s), respectively, the tracking module 60 can match the image or 3D geometry with the virtual models of the landmarks. Consequently, the tracking module 60 determines a spatial relationship between the landmarks being imaged and the preoperative 3D models, to provide a dynamic (e.g. real time or quasi real time) intraoperative tracking of the bones relative to the tools, in spite of tool portions and bone surfaces not being visible from the POV of the operator.

In an embodiment, the position and orientation of the surgical tool calculated by the tracking module 60 may be redundant over the tracking data provided by the robot driver 80 and robot arm sensors, if the robot arm 40 is used. However, the redundancy may assist in ensuring the accuracy of the tracking of the surgical tool. For example, the redundancy is used as a safeguard against disruption of the line of sight between the head-mounted tracking device 20 and the surgical site, for instance if the operator looks away. The redundancy may also allow the reduction of frequency of image processing for the surgical tool. Also, the tracking of the tool using the tracking module 60 may be used to detect any discrepancy between a calculated position and orientation of the surgical tool T through the sensors on the robot arm 40, and the actual position and orientation of the surgical tool. For example, an improper mount of the tool T into the chuck of the robot arm 40 could be detected from the output of the tracking module 60, when verified with the position and orientation from the robot driver 80 (e.g., obtained from the encoders on the robot arm 40). The operator may be prompted to verify the mount, via the interface I/F or head-mounted tracking device 20.

The camera 25 and/or tracking device 30 may continuously capture images, for the tracking module 60 to perform a continuous tracking of the objects. The terms video feed, image feed, video, may be used to describe the capture of images by the head-mounted tracking device 20, and optionally by the tracking device 30, and entails a capture of frames over a time period, in contrast to a single image capture. The frequency of capture may vary according to different factors. For example, there may be different phases during the surgical workflow, some in which the tracking requires a more dynamic update (i.e., higher frequency), and some in which tracking updates are less important. Another factor that may affect the image capture frequency is the fixed relation of the objects. For example, once the tracking module 60 identifies a landmark and tracks a bone from the images, the frequency capture by the camera 25 and/or tracking device 30 may be reduced if the bone is fixed or if no maneuvers are performed, if the bone alterations have not yet begun. Also, when both a tool and a bone are tracked, the frequency capture may be reduced when the tool and the bone are spaced from one another by a given distance, and increased as the proximity between the tool and the bone is increased. The tracking module 60 may drive the camera 25 and/or tracking device 90 in order to control the frequency. For example, the tracking module 60 may adapt the frequency using the surgical planning, e.g., anticipating upcoming steps in the workflow, etc. The tracking module 60 may consequently toggle between a lower frequency capture mode and a higher frequency capture mode, for example. The lower frequency capture mode may be in instances in which the tool is at a given distance from the bone, and is not driven to alter the bone. The lower frequency capture mode may also be operated when the objects are in a fixed relation relative to one another. Other modes are contemplated. The tracking module 60 may output the data directly on the interfaces I/F.

The augmented reality module 70 may be present in the CAS controller 50 and may produce an augmented reality (AR) output to the operator, for instance for display in the head-mounted tracking device 20. The augmented reality module 70 may also produce other types of outputs, including a virtual reality output. One example is an hologram view of the plates IN relative to the bone, along with trajectory FT, as a user is manipulating a tool (via the robot or in free hand mode), on the bone during the procedure. The augmented reality module 70 may provide its output to displays other than head-mounted tracking device 20. For example, the augmented reality module 70 may produce an output for display on monitors of the CAS system 10.

As seen in FIGS. 6-7, the floating GUIs shown as interface I/F may be projected or displayed onto the face shield 22 of the head-mounted tracking device 20, so as to be in the line of sight of the wearer, and/or may be duplicated or alternatively output on the interfaces I/F. In an embodiment, the head-mounted tracking device 20 projects the AR images in parts of the face shield 22 so as not to interfere with the line of sight between the wearer and bone or instrument, or to augment the objects being viewed, such as by adding axes to the bones, simulating the position of implants IM, or of the concealed tooling end of the tool T, among other possibilities.

In a variant, the wearer may also interface with the CAS controller 50 using the AR output from the head-mounted tracking device 20. For example, when looking away from the surgical site, or at given instances during the surgical workflow, the head-mounted tracking device 20 may display virtual touch zones, and may track with its camera 25 the wearer's arm reaching any such touch zone. The head-mounted tracking device 20 and CAS controller 50 may trigger an action based on the touch zone activation.

Accordingly, while the data provided by the augmented reality module 70 could be displayed on separate monitors or like interfaces, the display of the images in augmented reality may minimize the movements to be made by the wearer, increase the rapidity of access to information and/or provide information that may not otherwise be available.

Still referring to FIG. 1, the CAS controller 50 may have the robot driver module 80, if a robot arm 40 is present in the CAS system 10. The robot driver module 80 is tasked with powering or controlling the various joints of the robot arm 40. There may be some force feedback provided by the robot arm 40 to avoid damaging the bones. The robot driver module 80 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

Referring now to FIGS. 6 and 7, a contemplated use of the CAS system 10 is shown, as occurring during a bone model procedure in surgery, between femur and tibia, for ease of visual representation. FIGS. 6 and 7 show the intramedullary nail, but could also be used for the positioning of target plates as described above for FIGS. 4 and 5.

In the variant of FIG. 6, a surgeon or other operator has the head-mounted tracking device 20 with tracking technology as described above, with camera(s) 25 to perform point of view tracking. Optionally, though not shown, the tracking device 30 may be used as well. Tracking patterns may be on the implants, bones and/or tools, though they are optional, or could be elsewhere. The tracking using the video feed from the head-mounted tracking device 20 allows the CAS system 10 to limit obstructions as the operator usually maintains a direct unobstructed POV with the surgical site.

In the variant of FIG. 7, a surgeon or other operator has the head-mounted tracking device 20 with tracking technology as described above, with camera(s) 25 to perform point of view tracking, and with the robot arm 40 having a drill guide 40A to assist in locating the landmark locations such as locking holes 106 for the surgeon or other operator manipulating the tool T. Again, though not shown, the tracking device 30 may be used as well. Tracking patterns may be on the bones and/or tools, though they are optional, or could be elsewhere. The tracking using the video feed from the head-mounted tracking device 20 allows the CAS system 10 to limit obstructions as the operator usually maintains a direct unobstructed POV with the surgical site.

The CAS system 10 of FIGS. 6 and/or 7 may be used in the following manner. During surgery, with the head-mounted tracking device 20, the CAS system 10 may obtain a virtual model (e.g., three-dimensional) of the surgical implant IN, and optionally of the bone B, the virtual model of the surgical implant IN including at least one landmark (for instance, a locking hole 106) of the surgical implant IN, the at least one landmark being configured to be inside the bone B. The virtual models of the surgical implant IN and of the bone B may be pre-operative models resulting from different imaging modalities, may be manufacturer CAD models for implants, may be the result of a calibration, etc. This may include a step of calibrating a deformed implant IN intraoperatively, by digitizing points thereof, for example to obtained screwing axes of holes relative to a tracker device (e.g., EX, 204) or of a trackable part of the implant IN. Another approach to scanning would include the use of the cameras (e.g., head-mounted device 20, tracker device 30) to generate a 3D model. In some cases, the virtual models of the surgical implant IN and the bone B may be obtained using a pointer tool, notably to match a 3D virtual model with a cloud of points from a registration pointer. The CAS system 10 may track the surgical implant IN, via an optical non-radiographic tracking device (for instance, tracking device 30, head-mounted tracking device 20), as the surgical implant IN is inserted into the bone B. The CAS system 10 could use radiographic tracking to perform the procedure described herein, but the use of non-radiographic tracking simplifies the hardware requirements, and may contribute to streamlining the procedure. The CAS system may calculate a location of the at least one landmark relative to the bone B using the virtual model of the surgical implant IN and tracking data from the tracking of the surgical implant IN. The CAS system 10 may output the location of the at least one landmark relative to the bone B, in different forms including a drilling or fastener trajectory FT. This may take into consideration the presence of implants already in the patient, with the landmark(s) rendered available being free of interference with an implant. Stated differently, the CAS system 10 may output a drilling location and trajectory for a concealed hole to be drilled in the bone B as an entry point for a locking screw of like fastener used to secured the nail IN to the bone B by passing through a hole in the shaft 100. The CAS system 10 may hence detect stop conditions, to alarm the operator, and/or to stop the implant IN, for instance by indicating a proximity of the landmark location with a boundary of the bone. In parallel, the CAS system 10 may image or display the concealed at least one landmark relative to the bone B, such as on the head-mounted tracking device 20, e.g., in mixed reality on a face shield worn by the operator, or on other interfaces. In the variant of FIG. 7, the robot arm 40 may be controlled as a function of a position and orientation of the implant IN. Thus, the CAS system 10 may continuously output the location of the robot arm 40 (or of a tool supported by the robot arm 40, such as the illustrated cutting block).

As shown in FIGS. 3A and 3B, once the implant IN is fully inserted in the intramedullary canal 90 with the landmark locations of the locking holes 106, including their positions and orientations (e.g., trajectory), known, a tool such as a drill and a drilling guide may be used to insert the fasteners 110 through the bone B into the locking holes 106 to secure the implant IN to the bone B.

The present disclosure refers to the system 10 as performing continuous tracking. This means that the tracking may be performed continuously during discrete time periods of a surgical procedure. Continuous tracking may entail pauses, for example when the bone is not being altered. However, when tracking is required, the system 10 may provide a continuous tracking output, with any disruption in the tracking output triggering an alarm or message to an operator. The frequency of tracking may vary.

The CAS system 10 may generally be described as a system for tracking a surgical implant IN relative to a bone B in computer-assisted surgery. The system includes a processing unit and a non-transitory computer-readable memory communicatively coupled to the processing unit, which may, for instance, form part of the CAS controller 50. The non-transitory computer-readable memory comprises computer-readable program instructions executable by the processing unit for steps such as: obtaining a virtual model of the surgical implant IN, the virtual model including at least one landmark (for instance, a locking hole 106) of the surgical implant IN, the at least one landmark being configured to be inside the bone B; tracking the surgical implant IN, via an optical non-radiographic tracking device (for instance, tracking device 30), as the surgical implant IN is inserted into the bone B; calculating a location of the at least one landmark relative to the bone B using the virtual model of the surgical implant IN and tracking data from the tracking of the surgical implant IN; and outputting the location of the at least one landmark relative to the bone.

In a variant, the method and system describe herein operate in a traumatology setting, i.e., with limited pre-operative planning. In such a variant, the geometry of the implant IN may be known, including the position and orientation of the landmark location(s). Hence, by simply tracking the implant IN non-radiographically, the CAS system 10 may know its position and orientation in space, even without a model of the bone B. Therefore, by knowing the position and orientation in space of the implant IN (e.g., via extension EX), the CAS system 10 may output the trajectory and entry point of a drilling path, aligned with screw holes in the implant IN.

The CAS system 10 may be used in numerous other orthopedic procedures for different types of implants. Other examples include pelvic fracture plates, collarbone fracture plates, periprosthetic plates, among others.

1. A system for tracking a surgical implant relative to a bone in computer-assisted surgery, comprising:
a processing unit; and
a non-transitory computer-readable memory communicatively coupled to the processing unit and comprising computer-readable program instructions executable by the processing unit for:
obtaining a virtual model of the surgical implant, the virtual model including at least one landmark of the surgical implant, the at least one landmark being configured to be inside the bone;
tracking the surgical implant, via an optical non-radiographic tracking device, as the surgical implant is inserted into the bone;
calculating a location of the at least one landmark relative to the bone using the virtual model of the surgical implant and tracking data from the tracking of the surgical implant; and
outputting the location of the at least one landmark relative to the bone.

2. The system according to claim 1, wherein the computer-readable program instructions are executable by the processing unit for indicating a proximity of the at least one landmark with a boundary of the bone.

3. The system according to claim 2, wherein the computer-readable program instructions are executable by the processing unit for stopping the surgical implant when the at least one landmark is at the boundary of the bone.

4. The system according to claim 1, wherein the computer-readable program instructions are executable by the processing unit for imaging and displaying the location of the at least one landmark relative to the bone.

5. The system according to claim 4, wherein the computer-readable program instructions are executable by the processing unit for imaging and displaying the location of the at least one landmark relative to the bone in mixed reality on a face shield worn by the operator.

6. The system according to claim 1, including obtaining a model of the bone from pre-operative imaging.

7. The system according to claim 1, including obtaining the virtual model of the surgical implant from a manufacturer file.

8. The system according to claim 1, wherein the computer-readable program instructions are executable by the processing unit for controlling a robot arm as a function of a position and orientation of the surgical implant.

9. The system according to claim 8, wherein outputting the location of the at least one landmark relative to the bone includes outputting the location of the robot arm.

10. The system according to claim 1, including a head-mounted device with at least one camera for tracking the surgical implant.

11. The system according to claim 10, wherein the head-mounted device has a display for outputting data associated with the location of the at least one landmark relative to the bone, the data being output in mixed reality.

12. The system according to claim 1, wherein the surgical implant is an intramedullary nail configured to be inserted inside an intramedullary canal of the bone.

13. The system according to claim 12, wherein one or more landmark locations are locations of locking holes at proximal and distal ends of the intramedullary nail.

14. The system according to claim 13, wherein the computer-readable program instructions are executable by the processing unit for guiding a drilling tool to drill through the bone at the locations of the locking holes.

15. The system according to claim 1, wherein the surgical implant is at least one orthopedic plate configured to be positioned on the bone under soft tissue.

16. The system according to claim 15, wherein one or more landmark locations is a location of at least one locking hole on the orthopedic plate.

17. The system according to claim 16, wherein the computer-readable program instructions are executable by the processing unit for guiding a drilling tool to drill through the bone at the locations of the locking holes.

18. The system according to claim 13, wherein the computer-readable program instructions are executable by the processing unit for guiding a drilling tool to drill through the bone to interlock a pair of the at least one orthopedic plate.

19. The system according to claim 1, including obtaining a virtual model of the surgical implant using a pointer tool.

20. The system according to claim 1, including the optical non-radiographic tracking device.
